(19) United States
(12) Patent Application Publication
(10) Pub. No.: US 2024/0261572 A1
(43) Pub. Date: Aug. 8, 2024

(54) SYSTEMS AND METHODS FOR TRANSCUTANEOUS AURICULAR VAGAL NERVE STIMULATION TO ENHANCE MOTOR LEARNING, REHABILITATION, AND BCI APPLICATIONS

(71) Applicant: WASHINGTON UNIVERSITY, St. Louis, MO (US)

(72) Inventors: Eric Leuthardt, St. Louis, MO (US); Peter Brunner, St. Louis, MO (US); Kara Donovan, St. Louis, MO (US)

(21) Appl. No.: 18/566,890

(22) PCT Filed: Jun. 14, 2022
(86) PCT No.: PCT/US2022/033382

§ 371 Date: Dec. 4, 2023

Related U.S. Application Data
(60) Provisional application No. 63/210,991, filed on Jun. 15, 2021.

(51) Int. Cl. A61N 1/36; A61N 1/04; G16H 10/60; G16H 20/30

(52) U.S. Cl. CPC A61N 1/36031; A61N 1/0456; A61N 1/36025; A61N 1/36034; A61N 1/36036; G16H 10/60; G16H 20/30

(57) ABSTRACT

A method of enhanced motor learning is provided. The method includes stimulating a cutaneous distribution of a patient's vagus nerve within the patient's ear with a nerve stimulating signal. The method also includes assisting the patient in performing a physical activity while the patient's vagus nerve is stimulated. The method further includes monitoring one or more statistics of the patient during the physical activity.

### CROSS REFERENCE TO RELATED APPLICATIONS

This application claims the benefit of and priority to U.S. Provisional Patent Application No. 63/210,991, filed Jun. 15, 2021, entitled “TRANSCUTANEOUS AURICULAR VAGUS NERVE STIMULATION (TAVNS) TO ENHANCE MOTOR LEARNING, REHABILITATION, AND BCI APPLICATIONS,” which is hereby incorporated by reference in its entirety.

### STATEMENT REGARDING FEDERALLY SPONSORED RESEARCH OR DEVELOPMENT

This invention was made with government support under EB018783, EB026439, NS108916 and NS109103 awarded by the National Institutes of Health. The government has certain rights in the invention.

### BACKGROUND

The present disclosure generally relates to systems and methods of rehabilitating a motor function in a motor-impaired subject or enhancing motor function and performance in a normal subject.

Each year, over 50% of the nearly 800,000 Americans that suffer a stroke will experience prolonged upper limb motor deficits six months post-injury. While an abundance of stroke rehabilitation strategies already exists, there remain at least several barriers that limit patients' ability to improve. Many stroke rehabilitation strategies are dependent on the patient having considerable residual motor function, rendering them ineffective for those with severe hemiparesis or complete hemiplegia. Also, the vast majority of approaches do not use the patient's own neuronal activity, thus limiting their ability to maximize their central plasticity.

Brain-computer interface (BCI) therapy is well-suited for patients with substantial motor deficits because it provides targeted feedback based on their neuronal activity and does not require patients to initiate movement. While BCI systems can induce clinically significant functional improvements, BCI therapy is still a slow process and motor restoration is usually not complete. This is due to the reliance on endogenous plasticity-induced changes to generate recovery.

Vagus nerve stimulation (VNS) has emerged as a tool to promote and accelerate neuroplasticity in both healthy and injured brains, attributed in part to the release of plasticity-promoting neuromodulators at the cellular level. The vagus nerve is a mixed-fiber nerve that affects many upstream cortical and subcortical structures. Non-invasive transcutaneous auricular VNS (taVNS) has been demonstrated to improve post-stroke functional recovery. Despite encouraging preclinical and clinical results, the neural response to non-invasive VNS and the mechanism through which it affects functional motor recovery remain poorly understood in humans. This gap has limited the advancement of this therapeutic strategy. A well-characterized neural response to VNS will be critical in translating this approach in the chronic stroke population at scale.

### BRIEF DESCRIPTION

In a first aspect, a method of enhanced motor learning is provided. The method includes stimulating a cutaneous distribution of a patient's vagus nerve within the patient's ear with a nerve stimulating signal. The method also includes assisting the patient in performing a physical activity while the patient's vagus nerve is stimulated. The method further includes monitoring one or more statistics of the patient during the physical activity.

In a second aspect, a system for enhanced motor learning is provided. The system includes an electrical stimulation device including one or more electrodes. The electrical stimulation device is configured to provide an electrical current to a patient's vagus nerve with an electrical signal during a physical activity. The electrical signal is configured to stimulate a cutaneous distribution of a patient's vagus nerve within the ear with a nerve stimulating signal.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a system for providing vagal nerve stimulation to a patient in accordance with at least one embodiment.

FIG. 2 illustrates placement of electrodes for non-invasive transcutaneous vagus nerve stimulation using the system shown in FIG. 1.

FIG. 3 illustrates a process for providing vagal nerve stimulation using the system shown in FIG. 1.

FIG. 4 illustrates an example configuration of a client system shown in FIG. 3, in accordance with one embodiment of the present disclosure.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, devices and methods of treatment for rehabilitating a motor function in a motor-impaired subject in need. The motor impairment in the subject may result from any known injury, affliction, or disorder associated with motor impairment without limitation. Non-limiting examples of an injury, affliction, or disorder that may be rehabilitated using the systems and methods disclosed herein include a stroke such as a unilateral stroke, a spinal cord injury, a neuromuscular disorder, a traumatic brain injury, a limb amputation, a peripheral nerve injury, and any other associated with motor impairment.

In various aspects, the disclosed system for rehabilitating a motor function includes a brain-computer interface (BCI) configured to administer a motor function rehabilitation treatment to the subject and a non-invasive vagus nerve stimulation device to administer vagus nerve stimulation (VNS) to the subject during at least a portion of the motor function rehabilitation treatment. The VNS is configured to enhance a behavioral motor performance of the subject.

In some aspects, the transcutaneous stimulation of the auricular branch of the vagus nerve is implemented using a portable TENS (transcutaneous electrical nerve stimulation) unit connected to two ear clip electrodes positioned in an ear of the subject. Without being limited to any particular theory, the external ear is an effective position for non-invasive stimulation of the vagus nerve, where the auricular branch travels in the pinna of the ear. In one aspect, the ear clips used for the VNS treatment are positioned along the concha of the ear. In another aspect the device can be wholly configured to be affixed to the ear which include the electrode, power, electronics, and wearable form factor.

In various aspects, the BCI device may be any suitable device suitable for administering a motor function rehabilitation treatment to the motor-impaired subject without limitation. In some aspects, the BCI device may be an assistive BCI device configured to restore lost functions in the subject. Non-limiting examples of lost functions that may be restored using an assistive BCI device include communication in locked-in syndrome (e.g., as a result of amyotrophic lateral sclerosis), movements in paralysis, eating and drinking despite quadriplegia using robotic actuators and/or functional electrical stimulation systems. In other aspects, the BCI device may be a rehabilitative BCI device configured to foster neuroplasticity through manipulation or self-regulation of neurophysiological activity facilitating motor recovery using neurofeedback. In some aspects, the BCI device may translate electric, magnetic or metabolic brain activity into control signals of external devices that may replace, restore, enhance, supplement or improve the natural neural output, and thereby modify an ongoing interaction between the brain and its external or internal environment. Non-limiting examples of BCI devices suitable for use in the systems and methods disclosed herein are described in U.S. Pat. Nos. 9,730,816 and 10,596,014, as well as U.S. Patent Application Publication No. 2020/0188139, the contents of which are incorporated by reference herein in their entirety.

In various aspects, the BCI device may be any suitable device suitable for administering a motor function rehabilitation treatment to the motor-impaired subject without limitation. In some aspects, the non-invasive vagus nerve stimulation device may be any known non-invasive vagus nerve stimulation device without limitation. This could include electrical stimulation, vibration, or ultrasonic methods of activating the nerve. Non-limiting examples of non-invasive vagus nerve stimulation devices suitable for use in the systems and methods disclosed herein include a transcutaneous auricular vagus nerve stimulation (taVNS) device.

In various aspects, the VNS administered by the non-invasive vagus nerve stimulation device is configured to enhance a behavioral motor performance of the subject. Without being limited to any particular theory, VNS is thought to modify the brain activity of a subject in a manner such that the subject's motor learning and/or motor performance are enhanced. By way of non-limiting example, the VNS may increase neural activity in brain regions critical to motor learning based on anatomical projections from the nucleus tractus solitarius (NTS), where vagal fibers terminate. Non-limiting examples of anatomical projections from the NTS associated with motor learning include the amygdala, hippocampus, and prefrontal cortex. In various aspects, the increased neural activity may be characterized as increased power in orthodromic projections from the NTS within the alpha, beta, and/or gamma bands. Without being limited to any particular theory, power within the gamma band is thought to be associated with motor learning, and power within the alpha and beta bands is considered to be associated with motor function.

In various aspects, the vagus nerve stimulation is delivered as characterized by a VNS parameter comprising at least one of a stimulation frequency, a pulse-width, a current intensity, and any combination thereof. The VNS parameters may be any suitable value without limitation. In some aspects, the stimulation frequency ranges from about 10 Hz to about 50 Hz. In some aspects, the stimulation frequency is selected from 20 Hz, 30 Hz, or 40 Hz. In some aspects, the pulse-width ranges from about 100 us to about 500 μs. In some aspects, the pulse-width is selected from 100 μs, 250 μs, and 500 μs. In some aspects, the current intensity ranges from about 0.5 mA below a perceptual threshold to about the perceptual threshold, wherein the perceptual threshold comprises a current intensity sufficient to elicit a tingling sensation in the subject.

In various aspects, the systems disclosed above may be used in a method for rehabilitating a motor function in a motor-impaired subject in need. The method includes providing a brain-computer interface (BCI) and a non-invasive vagus nerve stimulation device similar to the devices described above. The method further includes administering a motor function rehabilitation treatment to the subject using the BCI as described above. The method further includes administering a vagus nerve stimulation (VNS) to the subject during at least a portion of the motor function rehabilitation treatment as described above, wherein the VNS is configured to enhance a behavioral motor performance of the subject.

The disclosed systems and methods combine non-invasive VNS with a BCI-driven rehabilitation technique to further enhance functional recovery in chronic stroke patients. In the examples herein, the optimal taVNS parameters to enhance motor learning were determined by recording invasive cortical physiology. Without being limited to any particular theory, taVNS is thought to contribute to elevated gamma power that increases brain activity and alertness, contributing to enhanced motor learning. The disclosed systems and methods provide a mechanism-driven approach to designing, optimizing, and clinically translating taVNS rehabilitation techniques to motor recovery in the setting of chronic stroke. Guided by encouraging findings from an invasively recorded non-human primate and behavioral human data as described in the examples herein, the optimal taVNS parameters to increase gamma activity in invasively monitored human subjects will be determined and the neurophysiology of the motor learning effect associated with taVNS will be defined. The effects of different stimulation parameters to determine the effects of stimulation frequency, pulse-width, and current intensity on the subjects' brain activity will be systematically evaluated.

Beyond motor rehabilitation these techniques of taVNS can be used to enhance motor function in neurologically normal human subjects. Specifically, taVNS can be performed in while doing motor tasks that require substantial repetition to master. Examples of these include athletic activities, musical activities, medical activities such as surgery, work related activities, or any complex movement. Examples of athletic activities include, but are not limited to, basketball, tennis, baseball, golf, running, volleyball, badminton, swimming, boxing, table tennis, skiing, ice skating, roller skating, cricket, rugby, pool, darts, football, bowling, ice hockey, surfing, martial arts, horse racing, snowboarding, skateboarding, cycling, archery, fishing, gymnastics, figure skating, rock climbing, sumo, wrestling, fencing, water skiing, jet skiing, weight lifting, scuba diving, snorkeling, wind surfing, sky diving, hang gliding, and bungee jumping. Examples of musical activities include, but are not limited to, playing a musical instrument, dancing, theatrical activities, ballet, opera, and singing. Examples of medical activities would include, but is not limited to, performance of a surgical procedure, clinical procedure, diagnostic procedure, and physical exam. Examples of a work related activity would include, but is not limited to, technical work task such as, but not limited to, electrical, plumbing or carpentry or other physical work, such as, but not limited to, construction, welding, roofing, and/or other physical labor.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

Optional” or “optionally” means that the subsequently described event or circumstance may or may not occur, and that the description includes instances where the event occurs and instances where it does not.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

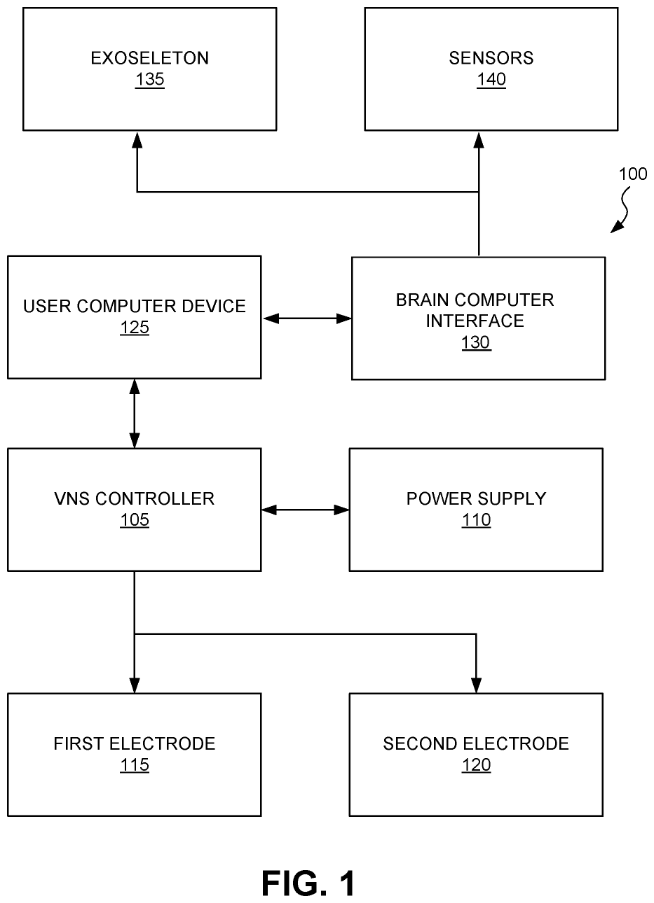

FIG. 1 illustrates a system 100 for providing vagal nerve stimulation to a patient in accordance with at least one embodiment.

A current trend in neuromodulation is the use of vagus nerve stimulation (VNS) as a means to promote neuroplasticity. The vagus nerve, which is comprised of 80% afferent fibers and 20% efferent fibers, is the main visceral sensory nerve and innervates many organs throughout the body. Stimulating the vagus nerve is typically performed using surgically implanted cuff electrodes—encircling the left vagus nerve within the carotid sheath—that are connected to a pulse generator implanted in the left side of the patient's chest. The left vagus nerve is used because it has fewer efferent fibers descending to the heart than the right vagus nerve, making it a safer site for stimulation. VNS has been proven effective as a treatment for intractable epilepsy and treatment-resistant depression, and has recently been investigated for several neurological injuries such as stroke and traumatic brain injury.

The vagus nerve is known to have a direct ascending projection to the nucleus tractus solitarius (NTS) which in turn activates the locus coeruleus (LC) and nucleus basalis (NB). The LC (located in the pons) and NB (located in the basal forebrain) are part of a neuromodulatory system with diffuse projections throughout cortical and subcortical areas. The LC contains noradrenergic neurons (norepinephrine, NE), and the NB contains cholinergic neurons (acetylcholine, ACh), both of which are known to be plasticity-promoting neuromodulators. The releases of NE and ACh are important in processes such as arousal, memory encoding, and task-related behavior, as well as processes requiring high attentional load. Thus, NE and ACh could have an important role in the mechanism of action for VNS-paired rehabilitation involving goal-directed behavior.

VNS stimulation triggers bursts of NE and ACh neuromodulator release causing changes in cortical plasticity. It is thought that these changes in cortical plasticity may lead to the therapeutic effect. Specifically, VNS has been shown to lead to reorganization of rat auditory and motor cortex, with increased cortical representations of VNS-paired tones or movements, respectively. This is further supported by lesion studies that have shown that depleting NE or ACh concentrations leads to blocked cortical plasticity and impaired learning. VNS has the capability to improve human recognition memory when administered at a moderate intensity. VNS has also been shown to improve retention on the Hopkins Verbal Learning Test when delivered during the memory consolidation phase, as well as to enhance working memory evidenced by reduced error rates on an executive functioning task.

While invasive VNS has been studied for several decades, non-invasive stimulation of the vagus nerve, specifically the auricular branch, which innervates the cymba concha and tragus regions of the outer ear, has emerged as an exciting non-invasive alternative. Transcutaneous auricular VNS (taVNS) provides clear benefits in eliminating the need for an invasive surgery and reducing the possible side effects which come with an implanted device. Several functional magnetic resonance imaging (fMRI) studies have demonstrated taVNS has central effects similar to invasive VNS. In comparison to sham earlobe stimulation, stimulating the left cymba concha has been shown to result in significant activation of the central vagal projections, such as the NTS and LC. Another fMRI study comparing the cymba concha and tragus as sites for taVNS found that both locations activated vagal projections, but only the cymba concha led to significant activations of the NTS and LC when compared to sham stimulation. Stimulating the vagus nerve via the outer ear has been investigated for many similar conditions as its invasive counterpart, such as epilepsy, depression, and tinnitus. Furthermore, similar to findings from invasive VNS, taVNS has also been shown to have cognitive benefits such as improved speech category learning and retention of non-native language tone categories, as well as enhanced associative memory in older adults.

The system 100 includes a VNS controller 105. The VNS controller 105 can be a computer device, such as a tablet, laptop, desktop, or other dedicated computer device including at least one processor in communication with at least one memory device. The VNS controller 105 can also include a user interface that that allows the VNS controller 105 to present information to a user and receive user inputs.

The VNS controller 105 is in communication with a power supply 110 configured to provide electrical stimulation. The VNS controller 105 can also be in communication with one or more electrodes, such as a first electrode 115 and a second electrode 120. The first electrode 115 and the second electrode 120 are configured to provide the electrical stimulation to the patient. In some embodiments, first electrode 115 and second electrode 120 are permanent, re-usable electrodes. In other embodiments first electrode 115 and second electrode 120 are disposable, single use electrodes. In still further embodiments, one or more of the first electrode 115 and the second electrode 120 are implanted in the patient to stimulate the vagus nerve. In additional embodiments, the first electrode 115 and the second electrode are temporarily attached to the patient's ear to stimulate the vagus nerve. In other embodiments, the first electrode 115 and the second electrode 120 provide electrical stimulation, vibration, or ultrasonic methods of activating the nerve,

In at least one embodiment, the VNS controller 105 is configured to provide treatment to the vagus nerve by electrically stimulation for a period of twenty minutes. In at least one embodiment, the attributes of the electrical stimulation are 20 Hz, 250 μs, and 0.4 mA. In other embodiments, the current can range between 0.4 and 8 mA. The attributes of the electrical stimulation stay the same throughout the treatment. In at least one further embodiment, the electrical stimulation is performed twice a day. In at least one embodiment, the attributes of the electrical stimulation are selected to maximize vagus somatosensory evoked potentials while avoiding perception of pain.

In the exemplary embodiment, the VNS controller 105 controls the output of the power supply 110 to provide the electrical stimulation via the first electrode 115 and the second electrode 120.

In some further embodiments, VNS controller 105 is in communication with one or more user computer devices 125. The user computer device 125 may provide information to the VNS controller 105, such as one or more attributes of the patient that may alter the electrical stimulation applied to the patient. Furthermore, the user computer device 125 may provide timing information to the VNS controller 105, such as when to apply the electrical stimulation. Moreover, the user computer device 125 can receive information from the VNS controller 105, such as what were the attributes of the electrical stimulation that was applied to the patient.

In still further embodiments, the user computer device 125 is also connected to a brain computer interface (BCI) 130. In these embodiments, the BCI 130 may be connected to an exoskeleton 135 and/or one or more sensors 140. In these embodiments, the goal is to combine non-invasive VNS with a non-invasive BCI-powered exoskeleton 135 to further enhance functional recovery in chronic stroke patients. The exoskeleton 135 assists the user/patient in moving one or more muscle groups. For example, the exoskeleton 135 is attached to the patient's arm and overlays their arm and attaches to their fingers and/or wrist. The exoskeleton 135 can receive messages from the patient via the BCI 130, which cause the exoskeleton 135 to move, thus moving the connected appendage of the patient. This can help those with limited motor control to help retrain their muscles and nerves.

The sensors 140 can be used to determine the optimal taVNS parameters to enhance motor learning by recording invasive cortical physiology. The taVNS contributes to elevated gamma power that will increase brain activity and alertness, contributing to enhanced motor learning. The integration of taVNS with a non-invasive BCI-powered exoskeleton in chronic stroke patients may improve their functional recovery.

In some embodiments, the sensors 140 include stereotactic electroencephalography (sEEG). The sensors 140 can then be used to monitor the effects of stimulation parameters on the subject's brain activity, especially during motor tasks. During these tasks, the sensors 140 may report the effect of stimulation frequency, pulse-width, and current intensity on the subject's brain activity. This may be used to find ideal parameters and/or adjust parameters to each individual subject. In at least one embodiment, parameter monitoring may be performed by monitoring a subject while they engage in a motor learning task paradigm (Serial Reaction Time Task, SRTT). The user computer device 125 can collect electrophysiological, behavioral, and kinematic data in order to fully characterize the effects of taVNS on motor learning. Other sensors 140 can include, but are not limited to, temperature, brain wave activity, galvanic response, blood pressure, heart rate, and/or any other attribute or statistic of the patient that is desired.

In some embodiments, the brain-computer interface 130 interacts with the exoskeleton 135 to harness the patient's own EEG activity. Based on each patient's uniquely identified mu- or beta-band activity, the exoskeleton 135 assists the patient in flexing and extending the fingers in coordination with changes in their own electrophysiological activity. However, patients with severe motor deficits face higher barriers to achieving clinically significant rehabilitation improvements. Thus, pairing taVNS with BCI therapy leads to accelerated plasticity and recovery. The user computer device 125 monitors the patient's brain activity and response to the taVNS stimulation and the interaction with the exoskeleton 135. In some embodiments, the user computer device 125 adjusts the output of the VNS controller 105 to maximize the patient's results and enhance the BCI therapy.

In some additional embodiments, the sensor 140 is capable of monitoring gamma band activity in the patient. The user computer device 125 may use the gamma band activity as a guideline of when to adjust the output of the VNS controller 105 to maximize the gamma band activity. 20-minutes of taVNS can induce a temporary post-stimulation increase in gamma activity. The decay of the stimulation-induced gamma augmentation indicates that taVNS-induced physiological changes are transient and return to normal within approximately five minutes after stimulation. Gamma band activity has been shown by several electrocorticographic (ECOG) studies to exhibit a strong correlation with certain aspects of motor, language, or cognitive function, and it is considered to represent local cortical processing. More specifically, increases in gamma power in the sensory cortex have been tied to increased attention and network engagement with a motor task. Since taVNS can cause a robust increase in this frequency band, these combination shows that taVNS is an effective approach for enhancing motor learning in chronic stroke patients by creating a more active overall brain state.

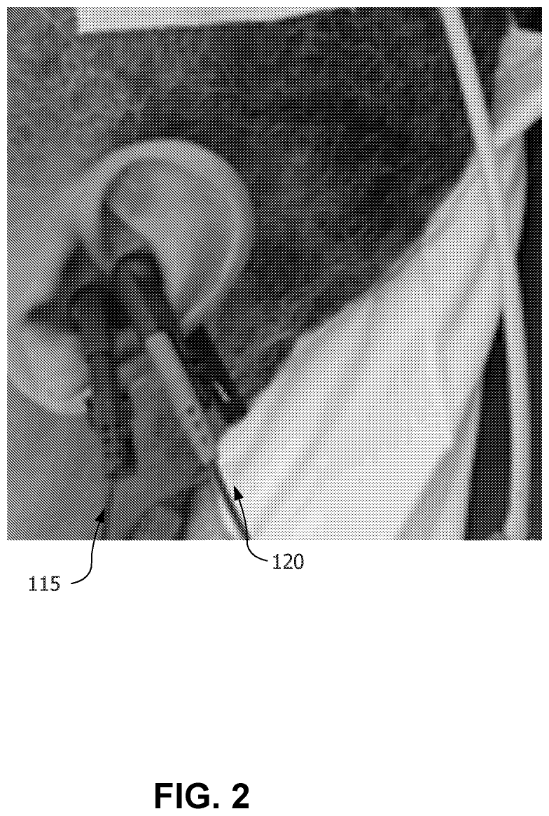

FIG. 2 illustrates placement of electrodes 115 and 120 (shown in FIG. 1) for non-invasive transcutaneous vagus nerve stimulation using the system 100 (shown in FIG. 1). In FIG. 2, the VNS controller 105 (shown in FIG. 1) is a part of a portable TENS (transcutaneous electrical nerve stimulation) unit. The TENS is connected to two the two electrodes 115 and 120.

In the exemplary embodiment, the first electrode 115 and the second electrode 120 are placed along the concha of the ear to stimulate the vagus nerve where the auricular branch travels in the pinna of the ear. In the exemplary embodiment, the first electrode 115 and the second electrode are attached to the patient's left ear.

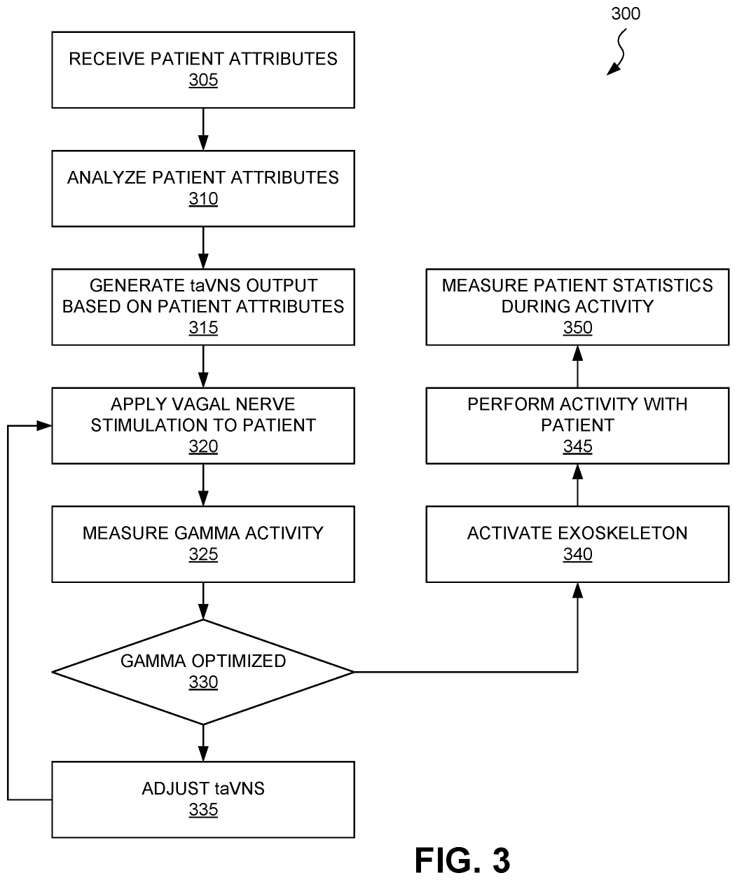

FIG. 3 illustrates a process 300 for providing vagal nerve stimulation and rehabilitating a motor function using the system 100 (shown in FIG. 1). In the exemplary embodiment, portions of process 300 are performed by a user computer device 125 (shown in FIG. 1), which may be, but is not limited to, a tablet, a laptop, a desktop, and/or and other computer device including at least one processor in communication with at least one memory device. Additionally, portions of process 300 are performed by the VNS controller 105 (shown in FIG. 1).

In the exemplary embodiment, the user computer device 125 receives 305 patient attributes. The patient attributes could be received 305 when the patient checks in or by retrieving the patient history. The patient attributes can include but are not limited to, height, weight, gender, heart rate, blood pressure, medical history, reasons for admittance, bloodwork results, vital statistics, presence/location of an aneurysm on vascular imaging, motor limitations, and other attributes. The patient attributes can further include CT (Computed tomography) imaging of stroke damaged nervous tissue. The patient attributes can be analyzed 310 to generate 315 the parameters for the taVNS stimulation based on the analyzed patient attributes.

In the exemplary embodiment, the user computer device 125 generates 315 appropriate parameters for taVNS stimulation of the patient based on historical analysis of a plurality of historical patients and their gamma activity to different taVNS parameters and also based on their patient attributes. In some embodiments, the user computer device 125 trains an artificial intelligence and/or machine learning model based on the historical data for the plurality of patients. In some of these embodiments, the model also includes historical information for the current patient based on previous treatments.

The healthcare provider may apply 320 vagal nerve stimulation to the patient using the system 100 (shown in FIG. 1). The healthcare provider attaches two electrodes (first electrode 115 and second electrode 120 (shown in FIG. 1)) to the concha of the left ear of the patient, as further shown in FIG. 2. The VNS controller 105 then provides a current through the electrodes 115 and 120. In the exemplary embodiment, the current has the following attributes: 20 Hz, 250 μs, and 8 mA. The current can range from 0.2 mA to 8 mA. The pulse width can range from 100 to 500 μs. The frequency can range from 20 to 40 Hz. Furthermore, other attributes of the current can change depending on other factors, such as the attributes of the patient. In some embodiments, the electrical stimulation remains at the same attributes during the entire period of stimulation. In other embodiments, the electrical stimulation is started at a lower current and the VNS controller 105 increases the current over time.

In the exemplary embodiment, the sensors 140 (shown in FIG. 1) measure the gamma activity of the patient in real-time. The user computer device 125 determines 330 if the detected gamma activity of the patient is optimized. In the exemplary embodiment, the detected gamma activity is optimized if the gamma activity is raised by a threshold amount, by a threshold percentage, to over a threshold value, or within a threshold range. In some embodiments, the model is used to determine the optimized gamma activity. If the gamma activity is not optimized, the user computer device 125 and/or the VNS controller 105 adjusts 335 the taVNS parameters and the new parameters are applied to the patient via VNS.

If the gamma activity is optimized, the user computer device 125 communicates with the brain computer interface 130 to activate the exoskeleton 135 (both shown in FIG. 1). In the exemplary embodiment, the user computer device 125 instructs the BCI 130 and the exoskeleton 135 to perform 345 an activity with the patient. For example, the exoskeleton 135 could be placed on the patient's arm and the activity is helping the patient perform exercises with that arm. During the activity, the VNS controller 105 is continuing to apply 320 vagal nerve stimulation to the patient. During the activity, the user computer device 125 and the sensors 140 measure 350 the patient's statistics. The patient statistics may be analyzed to determine the patient's progress as well as a notification of when the patient should discontinue the treatment session.

In at least one example, the activity takes up to twenty minutes. During that time, the VNS controller 105 applies 320 VNS to the patient. When the activity is over, the VNS is discontinued and the patient is disconnected from the exoskeleton 135. In other embodiments, the exercise treatment only takes a few minutes, but the length of the treatment increases incrementally over time, where the patient works up to being able to handle longer exercise treatments. In other embodiments, the patient may receive treatment for one side of the body, the other, or both either serially or simultaneously. In some embodiments, the patient's statistics are monitored after the VNS is discontinued to monitor how the patient's statistics return to baseline.

The exoskeleton 135 may be configured to provide assistance to the patient during the exercise treatment. In some embodiments, the exoskeleton 135 provides motorized assistance in movements to the patient. For example, the exoskeleton 135 could help the patient move their hand and/or fingers by pushing or otherwise assisting the movements. In other embodiments, the exoskeleton 135 provides resistance to the patient's movements to increase the amount of exertion required to make those movements. In still further embodiments, the patient may perform the exercise treatment without an exoskeleton 135, and just perform exercises while receiving VNS. Other forms that can guide the physical activity could be wearable, virtual reality, and software visualization presented on a tablet, mobile phone, or personal computer.

In some embodiments, the activity is a motor learning task which may be a version of a Serial Reaction Time Task (SRTT). This task uses stimulus presentation, while being integrated with the BCI 130 to synchronously acquire electrophysical and behavioral data. In one example, the SRTT has the patient is asked to place the fingers of their dominant hand on a keyboard, and to respond as quickly and accurately as possible with a keyboard press to a corresponding visual cue. Patients will also be instructed to maintain gaze on the screen. If a patient fail to respond to a visual cue within 1500 ms, they will be reminded to respond faster. Each response is followed by a random 200-500 ms-long inter-trial interval (ITI). Subjects will perform multiple trials with repeating sequences and random sequences interleaved and counterbalanced throughout the experiment. Throughout the activity, the patients will be blinded from the underlying sequence and stimulation structure. Which will prevent subjects from being distracted or biased by the task design and will enable testing of implicit motor learning. To prevent the subject from anticipating the visual cue onset, catch trials with a 1000 ms-long ITI will be included. The catch trials will also ensure the subjects remain attentive and allow us to assess selective responsiveness.

To keep the patients engaged and motivated throughout each session, they will receive feedback on their accuracy, and they will be alerted if they respond too slowly. In one example, each activity consists of seven blocks with twelve trials each, where a trial is defined as one visual cue. Kinematic data can be obtained through a data glove or other sensor 140 and consist of finger flexion and extension measured from three locations per finger on the patient's dominant hand, which they will use to complete the task.

During this task, the sensors 140 will collect electrophysiological, behavioral, and kinematic data in order to fully determine the effect of taVNS on motor learning. To ensure the quality of data, the gaze fixation data will enable verification that the patients were paying attention and will also be used to measure subjects' implicit learning by assessing if they start to accurately predict the location of the next visual cue during the repeated sequences. The primary behavioral metrics will include reaction time and error rate. The primary electrophysiological metrics will be power measures in our frequency bands of interest: alpha, beta, and gamma. Kinematic data will be analyzed to assess predictive finger movements prior to the cue being presented. To correlate the behavioral and electrophysiological metrics, the user computer device 125 will perform a linear regression of the behavioral outcomes (reaction time and accuracy) against primary electrophysiological metric (broadband gamma power).

In the exemplary embodiment, the user computer device 125 trains brain models using historical data from a plurality of patients. Then the user computer device 125 receives information about an individual patient to train a brain model of that patient. In some embodiments, the user computer device 125 trains the brain model of the patient over a plurality of treatments based on how the patient's brain reacts to different levels of stimulation during the treatments. The user computer device 125 can use the brain model to fine tune the settings of the VNS controller 105 to provide optimal gamma activity. In some embodiments, the brain models are based on Tl-weighted structural magnetic resonance images (MRI) and/or computed tomography (CT) scans.

The sensor information will also be filtered with highpass/lowpass/notch filtered to remove environmental noise. In some embodiments, time periods with artifacts or pathological activity defined as greater than 10 seconds of artifact or one or more spikes larger than a five-fold increase in the magnitude of the baseline signal will be rejected. Band-limited amplitude timeseries can be extracted by convolving a Gabor wavelet of the desired frequency or frequency band with the preprocessed signal. Power envelopes for each signal can be calculated by squaring the absolute value of the amplitude timeseries. The overall power for a segment of the sensor recordings can be estimated by calculating the mean of the power envelope for that segment. Power in the resting period can be calculated for delta (1-3 Hz), theta (4-7 Hz) mu (8-12 Hz), beta (13-30 Hz), and broadband gamma bands (70-170 Hz).

In the exemplary embodiment, the taVNS will result in increased gamma rhythm power in cortical and subcortical sites that share orthodromic connections with the nucleus tractus solitarius (NTS). Specifically, a metric of success will be statistically significant (corrected, p<0.05) changes in high frequency gamma (70-100 Hz) power in key upstream regions from the locus coeruleus (LC) which will include the hippocampus, amygdala, prefrontal cortex, and primary somatosensory cortex. Further, the user computer device 125 will determine optimal parameters for this central effect based on observed data, both historical and real-time. Also, based on the expectation that VNS works by increasing arousal, and considering that arousal is a strong predictor for attention and memory, there is an expectation to see a stronger phasic GSR (biomarker for emotional arousal) during active taVNS as compared to sham or baseline.

In some embodiments, the differences in the gamma rhythm signal power are detected when comparing taVNS against baseline control. For example, the gamma power at baseline could be 0.0041+0.0053 μV2/s, and the gamma power during VNS would be 0.0059+0.0078 μV2/s(78), which corresponds to an effect size of 0.26.

In the exemplary embodiment, the VNS controller 105 stimulates a patient's vagus nerve with an electrical signal to increase gamma activity to enhanced motor learning. The electrical signal is applied during physical activity of the patient. One or more sensors 140 monitor one or more statistics of the patient during the physical activity. A user computer device 125 determines a current level of gamma activity for the patient. The user computer device 125 adjusts one or more parameters of the electrical signal to change the current level of gamma activity. In some embodiments, the physical activity is assisted by a powered exoskeleton 135 that assists and/or guides the movements of the patient. The patient previously suffered a stroke. The stimulation is transcutaneous stimulation of the vagus nerve. The stimulation is provided via a first electrode 115 and a second electrode 120. The first electrode 115 and the second electrode 120 are attached to the concha of the patient's ear. The first electrode 115 and the second electrode 120 are attached to the patient's left ear. The stimulation is provided to the auricular branch of the vagus nerve where the vagus nerve travels in the pinna of the ear. Other forms that can guide the physical activity could be wearable, virtual reality, and software visualization presented on a tablet, mobile phone, or personal computer.

In some embodiments, the user computer device 125 receives a plurality of patient attributes associated with the patient. The user computer device 125 analyzes the plurality of patient attributes. The user computer device 125 determines one or more parameters of the electrical signal based on the analyzed patient attributes. In other embodiments, the user computer device 125 receives a plurality of monitored statistics of the patient from previous treatments. The user computer device 125 analyzes the plurality of monitored statistics. The user computer device 125 determines one or more parameters of the electrical signal based on the analyzed monitored statistics.

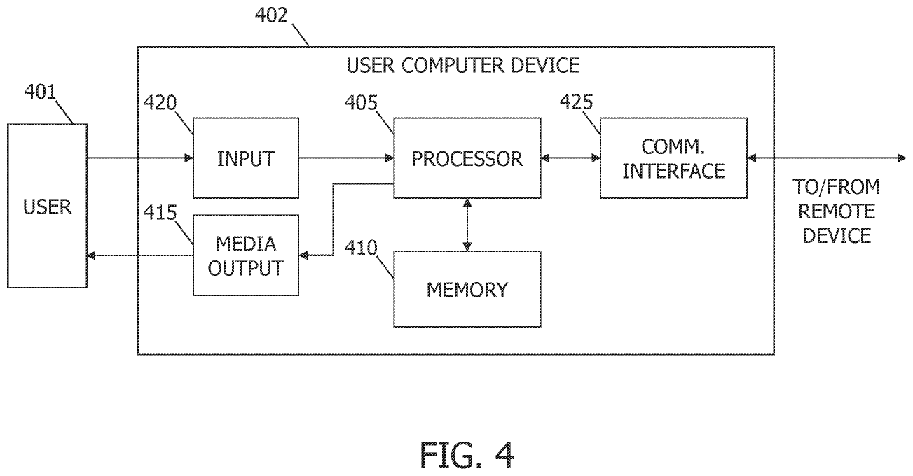

FIG. 4 illustrates an example configuration of a client system shown in FIG. 3, in accordance with one embodiment of the present disclosure. User computer device 402 is operated by a user 401. User computer device 402 may include, but is not limited to, VNS controller 105 and user computer device 125 (both shown in FIG. 1). User computer device 402 includes a processor 405 for executing instructions. In some embodiments, executable instructions are stored in a memory area 410. Processor 405 may include one or more processing units (e.g., in a multi-core configuration). Memory area 410 is any device allowing information such as executable instructions and/or transaction data to be stored and retrieved. Memory area 410 may include one or more computer-readable media.

User computer device 402 also includes at least one media output component 415 for presenting information to user 401. Media output component 415 is any component capable of conveying information to user 401. In some embodiments, media output component 415 includes an output adapter (not shown) such as a video adapter and/or an audio adapter. An output adapter is operatively coupled to processor 405 and operatively coupleable to an output device such as a display device (e.g., a cathode ray tube (CRT), liquid crystal display (LCD), light emitting diode (LED) display, or “electronic ink” display) or an audio output device (e.g., a speaker or headphones). In some embodiments, media output component 415 is configured to present a graphical user interface (e.g., a web browser and/or a client application) to user 401. A graphical user interface may include, for example, patient attributes or the attributes of the electrical stimulation. In some embodiments, user computer device 402 includes an input device 420 for receiving input from user 401. User 401 may use input device 420 to, without limitation, select to apply the electrical stimulation to the patient. Input device 420 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 415 and input device 420.

User computer device 402 may also include a communication interface 425, communicatively coupled to a remote device such as a VNS controller 105 or a user computer device 125. Communication interface 425 may include, for example, a wired or wireless network adapter and/or a wireless data transceiver for use with a mobile telecommunications network.

Stored in memory area 410 are, for example, computer-readable instructions for providing a user interface to user 401 via media output component 415 and, optionally, receiving and processing input from input device 420. The user interface may include, among other possibilities, a web browser and/or a client application. Web browsers enable users, such as user 401, to display and interact with media and other information typically embedded on a web page or a website provided by a server. A client application allows user 401 to interact with, for example, VNS controller 105. For example, instructions may be stored by a cloud service and the output of the execution of the instructions sent to the media output component 415.

The methods and systems described herein may be implemented using computer programming or engineering techniques including computer software, firmware, hardware, or any combination or subset thereof, wherein the technical effects may be achieved by performing at least one of the following steps: a) stimulating the cutaneous distribution of a patient's vagus nerve within the ear with a nerve stimulating signal; b) assisting the patient in performing a physical activity while the patient's vagus nerve is stimulated; c) monitoring one or more statistics of the patient during the physical activity; d) determining the movement performance for the patient; e) adjusting one or more parameters of the electrical signal to change the movement performance; f) the physical activity is assisted by a powered exoskeleton that assists and/or guides the movements of the patient; g) the physical activity is assisted by a virtual reality headset; h) the physical activity is assisted by a software visualization on a mobile computer device; i) the patient previously suffered a stroke; j) the patient previously suffered a spinal cord injury; k) the patient previously suffered a traumatic brain injury; l) the patient previously suffers from multiple sclerosis; m) patient is neurologically normal and wants to enhance motor performance of a specific task; n) the specific task is an athletic activity; o) the specific task is a musical activity; p) the specific task is a surgical activity; q) the specific task includes complex motor movement; r) the stimulation is electrical stimulation of the vagus nerve; s) the stimulation is vibrotactile stimulation of the vagus nerve; t) the stimulation is provided via a first electrode and a second electrode, wherein the first electrode and the second electrode are attached to the concha of the patient's ear; u) the first electrode and the second electrode are attached to the patient's left ear. v) the stimulation is provided to the auricular branch of the vagus nerve where the vagus nerve travels in the pinna of the ear; w) receiving a plurality of patient attributes associated with the patient; x) analyzing the plurality of patient attributes; y) determining one or more parameters of the electrical signal based on the analyzed patient attributes; z) receiving a plurality of monitored statistics of the patient from previous treatments; aa) analyzing the plurality of monitored statistics; and bb) determining one or more parameters of the electrical signal based on the analyzed monitored statistics.

A computer program of one embodiment is embodied on a computer-readable medium. In an example, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

A processor or a processing element may be trained using supervised or unsupervised machine learning, and the machine learning program may employ a neural network, which may be a convolutional neural network, a deep learning neural network, a reinforced or reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, object statistics and information, traffic timing, previous trips, and/or actual timing. The machine learning programs may utilize deep learning algorithms that may be primarily focused on pattern recognition, and may be trained after processing multiple examples. The machine learning programs may include Bayesian Program Learning (BPL), voice recognition and synthesis, image or object recognition, signal processing, optical character recognition, and/or natural language processing—either individually or in combination. The machine learning programs may also include natural language processing, semantic analysis, automatic reasoning, and/or machine learning.

Supervised and unsupervised machine learning techniques may be used. In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. In one embodiment, machine learning techniques may be used to determine brain responses to stimuli such as VNS settings.

Based upon these analyses, the processing element may learn how to identify characteristics and patterns that may then be applied to analyzing image data, model data, and/or other data. For example, the processing element may learn, to identify brain responses to stimuli and the VNS settings for different patients to provide optimal gamma activity. The processing element may also learn how to identify trends that may not be readily apparent based upon collected traffic data, such as trends that identify when gamma activity will spike or decline.

The exemplary systems and methods described and illustrated herein therefore provide VNS treatments for improving the effectiveness of motor learning treatments.

The computer-implemented methods and processes described herein may include additional, fewer, or alternate actions, including those discussed elsewhere herein. The present systems and methods may be implemented using one or more local or remote processors, transceivers, and/or sensors (such as processors, transceivers, and/or sensors mounted on vehicles, stations, nodes, or mobile devices, or associated with smart infrastructures and/or remote servers), and/or through implementation of computer-executable instructions stored on non-transitory computer-readable media or medium. Unless described herein to the contrary, the various steps of the several processes may be performed in a different order, or simultaneously in some instances.

Additionally, the computer systems discussed herein may include additional, fewer, or alternative elements and respective functionalities, including those discussed elsewhere herein, which themselves may include or be implemented according to computer-executable instructions stored on non-transitory computer-readable media or medium.

In the exemplary embodiment, a processing element may be instructed to execute one or more of the processes and subprocesses described above by providing the processing element with computer-executable instructions to perform such steps/sub-steps, and store collected data (e.g., trust stores, authentication information, etc.) in a memory or storage associated therewith. This stored information may be used by the respective processing elements to make the determinations necessary to perform other relevant processing steps, as described above.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

Although specific features of various embodiments may be shown in some drawings and not in others, this is for convenience only. In accordance with the principles of the systems and methods described herein, any feature of a drawing may be referenced or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a programmable logic unit (PLU), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of enhanced motor learning, the method comprising:
stimulating a cutaneous distribution of a patient's vagus nerve within the patient's ear with a nerve stimulating signal;
assisting the patient in performing a physical activity while the patient's vagus nerve is stimulated; and
monitoring one or more statistics of the patient during the physical activity.

2. The method of enhanced of claim 1 further comprising:
determining movement performance for the patient; and
adjusting one or more parameters of the nerve stimulating signal to change the movement performance.

3. The method of claim 1, wherein the physical activity is assisted by a powered exoskeleton that assists and/or guides the physical activity of the patient.

4. The method of claim 1, wherein the physical activity is assisted by a virtual reality headset.

5. The method of claim 1, wherein the physical activity is assisted by a software visualization on a mobile computer device.

6. The method of claim 1, wherein the patient previously suffered a stroke.

7. The method of claim 1, wherein the patient previously suffered a spinal cord injury.

8. The method of claim 1, wherein the patient previously suffered a traumatic brain injury.

9. The method of claim 1, wherein the patient previously suffers from multiple sclerosis.

10. The method of claim 1, wherein the patient is neurologically normal and wants to enhance motor performance of a specific task.

11. The method of claim 11, wherein the specific task is an athletic activity.

12. The method of claim 11, wherein the specific task is a musical activity.

13. The method of claim 11, wherein the specific task is a surgical activity.

14. The method of claim 11, wherein the specific task includes complex motor movement.

15. The method of claim 1, wherein the stimulation is electrical stimulation of the vagus nerve.

16. The method of claim 1, wherein the stimulation is vibrotactile stimulation of the vagus nerve.

17. The method of claim 16, wherein the stimulation is provided via a first electrode and a second electrode, wherein the first electrode and the second electrode are attached to the concha of the patient's ear.

18. The method of claim 17, wherein the first electrode and the second electrode are attached to the patient's left ear.

19. The method of claim 17, wherein the stimulation is provided to the auricular branch of the vagus nerve where the vagus nerve travels in the pinna of the ear.

20. The method of claim 1 further comprising:
receiving a plurality of patient attributes associated with the patient;
analyzing the plurality of patient attributes; and
determining one or more parameters of the nerve stimulating signal based on the analyzed patient attributes.

21. The method of claim 1 further comprising:
receiving a plurality of monitored statistics of the patient from previous treatments;
analyzing the plurality of monitored statistics; and
determining one or more parameters of the nerve stimulating signal based on the analyzed monitored statistics.

22. A system for enhanced motor learning, the system comprising an electrical stimulation device including one or more electrodes, wherein the electrical stimulation device is configured to provide an electrical current to a patient's vagus nerve with an electrical signal during a physical activity, wherein the electrical signal is configured to stimulate a cutaneous distribution of a patient's vagus nerve within the patient's ear with a nerve stimulating signal.
